(19) United States
(12) Patent Application Publication
(10) Pub. No.: US 2024/0051969 A1
(43) Pub. Date: Feb. 15, 2024

(54) ROR-GAMMA INHIBITORS FOR THE TREATMENT OF ROR-GAMMA-DEPENDENT CANCER

(71) Applicant: DIACCURATE, PARIS (FR)

(72) Inventors: JONATHAN LEVY, PARIS (FR); ANTOINE PAU, PARIS (FR); DOMINIQUE BRIDON, SAN FRANCISCO, CA (US)

(21) Appl. No.: 18/265,267

(22) PCT Filed: Dec. 7, 2021
(86) PCT No.: PCT/EP2021/084617

§ 371 Date: Jun. 5, 2023

(30) Foreign Application Priority Data
Dec. 7, 2020 (EP) 20306509.9

(51) Int. Cl. C07D 495/20; A61K 45/06; A61P 35/00

(52) U.S. Cl. CPC C07D 495/20; A61K 45/06; A61P 35/00

(57) ABSTRACT

The present invention relates to particular ROR gamma inhibitors for use for treating RORγ dependent cancer or metastasis, particularly prostate cancer, such as castration-resistant prostate cancer.

The present invention relates to RORγ inhibitors and pharmaceutical compositions containing such RORγ inhibitors for use in the treatment of RORγ dependent cancer. Particularly, the present invention relates to RORγ inhibitors and pharmaceutical compositions containing such RORγ inhibitors for use in the treatment of prostate cancer and metastasis, such as castration-resistant prostate cancer.

### BACKGROUND

Cancer is a leading cause of death in industrial countries. Various different treatment methods have been developed, such as chemotherapy, radiation therapy, and hormone deprivation therapy. However, there is no 100% effective cure to these diseases. One of the reasons current cancer treatment methods do not result in eradication of the cancerous tissue in afflicted individuals is through the development of drug resistance by the cancerous cells. Patients who exhibit drug resistance to a particular cancer drug will have tumors that no longer react to the drug and can continue growing despite continued treatment.

For example, prostate cancer is the second most frequently diagnosed cancer and the sixth leading cause of cancer death in males worldwide. Androgen deprivation therapy (ADT) is used to treat advanced prostate cancer with some initial benefits. However, although ADT is initially effective, disease progression to castration-resistance prostate cancer (CRPC) eventually occurs in almost all patients. While there are some treatment modalities for CRPC, resistance occurs after a few months and CRPC is currently incurable.

There is a need for improved methods of treating cancers, particularly RORγ dependent cancers, such as prostate cancer.

### SUMMARY OF THE INVENTION

It is an object of the present invention to provide a compound of formula (I), or a pharmaceutically acceptable salt thereof,

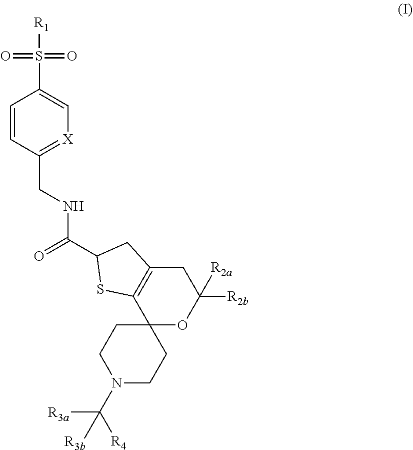

wherein
n is 0, 1 or 2,
X is independently —N— or —CH—,
R1 is —C1-3 alkyl,
R2a and R2b are independently —H or —CH3,
R3a and R3b are independently —H, CH3 or —CH2CH3,
R4 is

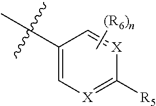

or thiazolyl, optionally substituted with —CF3,
R5 is halo, —CN, —CF3, oxadiazolyl, or oxadiazolyl, optionally substituted with CH3,
R6 is —H, —OMe, halo, —CH3 or —CF3,
for use in the treatment of RORγ dependent cancer or metastasis, particularly prostate cancer or metastasis.

In a particular embodiment, the compound is of formula (II),

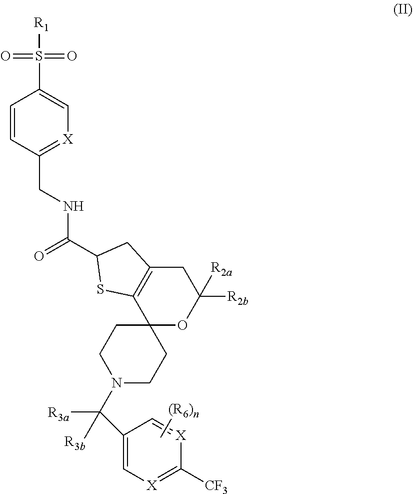

wherein
n is 0 or 1,
X is independently —N— or —CH—,
R1 is —C1-3 alkyl,
R2a and R2b are independently —H or —CH3,
R3a and R3b are independently —H, CH3 or —CH2CH3,
R6 is —H, —OMe, halo, —CH3 or —CF3.

Preferably, the compound is of formula (III),

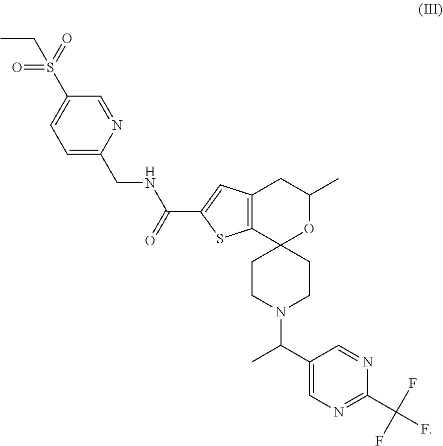 [SMILES: CCS(=O)(=O)c1ccc(CNC(=O)c2cc3c(s2)C2(CCN(C(C)c4cnc(C(F)(F)F)nc4)CC2)OC(C)C3)nc1]

It is another object of the present invention to provide a pharmaceutical composition comprising a compound of formula (I), (II) or (III) or a pharmaceutically acceptable salt thereof, in combination with one or more pharmaceutically acceptable carriers, diluents and/or excipients, for use in the treatment of RORγ dependent cancers or metastasis, particularly prostate cancer or metastasis.

It is a further object of the present invention to provide a kit for use in the treatment of RORγ dependent cancers or metastasis, particularly prostate cancer or metastasis, comprising a compound of formula (I), (II) or (III) or a pharmaceutically acceptable salt thereof, and an anticancer drug.

### DETAILED DESCRIPTION OF THE INVENTION

The present invention relates to the use of a RORγ inhibitor according to formula (I), particularly a RORγ inhibitor of formula (II), such as RORγ inhibitor according to formula (III), for treating RORγ dependent cancers or metastasis, particularly prostate cancer or metastasis, such as castration-resistant prostate cancer. Particularly, the present invention relates to a method of treatment of prostate cancer by administration to a subject in need thereof of a compound according to formula (III). The compounds of the present invention present particular interest for treating prostate cancer. Indeed, the compounds of the present invention has great drug candidate properties including but not limited to great affinity for the ROR gamma receptor, with high selectivity vs. RORα, RORβ and other nuclear hormone receptors, great potency and overall efficacy. Furthermore, the compounds of the present invention exhibit excellent drug-like properties including great oral bioavailability, great first-pass metabolism, good half-lives, and great pharmacokinetic properties including but not limited to good absorption, clearance, excretion and volume of distribution.

### Definitions

The terms “subject”, “patient” or “individual” are used herein interchangeably to include a human or animal. For example, the animal subject may be a mammal, a primate (e.g., a monkey), a livestock animal {e.g., a horse, a cow, a sheep, a pig, or a goat), a companion animal {e.g., a dog, a cat), a laboratory test animal {e.g., a mouse, a rat, a guinea pig, a bird), an animal of veterinary significance, or an animal of economic significance. Preferably, the “subject” is a human who is awaiting the receipt of, or is receiving medical care or was/is/will be the object of a medical procedure, or is monitored for the diagnosis or the development of a disease. In the context of the present invention, a “subject” or a “patient” refers more particularly to an adult man, susceptible to develop or developing a prostate cancer.

As used herein, the terms “RORγ dependent cancer(s) or metastasis” or “RORγ sensitive cancer(s) or metastasis” refer to cancer(s) or metastasis that involve IL-23-responsive, RORγ or RORyt, and IL-17-expressing cells. Examples of RORγ dependent cancers include prostate cancer, breast cancer, particularly Triple-Negative Breast Cancer (TNBC), pancreatic cancer, gastric cancer, hepatocellular carcinoma (HCC); lung cancer, liver cancer, ovarian cancer, endometrial cancer, bladder cancer, colon cancer, lymphoma, and glioma.

As used herein, the terms “prostate cancer” or “prostate cancer cell” refer to a cancer cell or cells that reside in prostate tissue. The prostate cancer can be benign, malignant, or metastatic. The prostate cancer can be androgen-insensitive, hormone-resistant, or castrate-resistant. The prostate cancer can be “advanced stage prostate cancer” or “advanced prostate cancer.” Advanced stage prostate cancer includes a class of prostate cancers that has progressed beyond early stages of the disease. Typically, advanced stage prostate cancers are associated with a poor prognosis. Types of advanced stage prostate cancers include, but are not limited to, metastatic prostate cancer, drug-resistant prostate cancer such as anti-androgen-resistant prostate cancer (e.g., enzalutamide-resistant prostate cancer, abiraterone-resistant prostate cancer, bicalutamide-resistant prostate cancer, etc.), taxane-resistant prostate cancer (e.g., docetaxel-resistant prostate cancer) and the like, hormone refractory prostate cancer, castration-resistant prostate cancer (CRPC), metastatic castration-resistant prostate cancer, AR-V7-induced drug-resistant prostate cancer, AKR1C3-induced drug-resistant prostate cancer, and combinations thereof. In some instances, the advanced stage prostate cancers do not generally respond, or are resistant, to treatment with one or more of the following conventional prostate cancer therapies: enzalutamide, abiraterone, bicalutamide, and docetaxel. Compounds, compositions, and methods of the present invention are provided for treating prostate cancer, such as advanced stage prostate cancer, including any one or more (e.g., two, three, four, five, six, seven, eight, nine, ten, or more) of the types of advanced stage prostate cancers disclosed herein.

In the context of the invention, the terms “treatment”, “treat” or “treating” are used herein to characterize a therapeutic method or process that is aimed at (1) slowing down or stopping the progression, aggravation, or deterioration of the symptoms of the disease state or condition to which such term applies; (2) alleviating or bringing about ameliorations of the symptoms of the disease state or condition to which such term applies; and/or (3) reversing or curing the disease state or condition to which such term applies. Particularly, in the context of prostate cancer, if the clinician notes objective changes, such as reducing the number of cancer cells, the growth of the cancer cells, the size of cancer tumors, or the resistance of the cancer cells to another cancer drug, then treatment of cancer has also been beneficial.

As used herein, the phrase “reversing cancer cell resistance” includes altering or modifying a cancer cell that is resistant to anticancer drug therapy so that the cell is no longer resistant to anticancer drug therapy.

As used herein, the phrase “reducing cancer cell resistance” includes increasing the therapeutic activity of an anticancer drug towards cancer cells that are, or previously were, resistant to anticancer drug therapy.

As used herein, the phrase “enhancing the therapeutic effects” includes any of a number of subjective or objective factors indicating a beneficial response or improvement of the condition being treated as discussed herein. For example, enhancing the therapeutic effects of an anticancer drug such as an anti-androgen drug (e.g., enzalutamide, abiraterone, or bicalutamide) or a chemotherapeutic agent such as tamoxifen or a taxane (e.g., docetaxel) includes reversing or reducing cancer cell resistance and/or sensitizing a drug-resistant cancer to anticancer drug therapy. Also, for example, enhancing the therapeutic effects of an anticancer drug includes altering drug-resistant cancer cells so that the cells are not resistant to the anticancer drug. Also, for example, enhancing the therapeutic effects of an anticancer drug includes additively or synergistically improving or increasing the activity of the anticancer drug. In some embodiments, the enhancement includes a one-fold, two-fold, three-fold, five-fold, ten-fold, twenty-fold, fifty-fold, hundred-fold, or thousand-fold increase in the therapeutic activity of an anticancer drug used to treat cancer.

The terms “quantity,” “amount,” and “dose” are used interchangeably herein and may refer to an absolute quantification of a molecule.

As used herein, the terms “active principle”, “active ingredient” and “active pharmaceutical ingredient” are equivalent and refer to a component of a pharmaceutical composition having a therapeutic effect.

As used herein, the term “therapeutic effect” refers to an effect induced by an active ingredient, or a pharmaceutical composition according to the invention, capable to prevent or to delay the appearance of a disease, or to cure or to attenuate the effects of a disease.

A “therapeutically effective amount” or “efficient concentration” refers to mean levels or amount of substance that is aimed at, without causing significant negative or adverse side effects to the target, delaying or preventing the onset of a RORγ dependent cancer, particularly a cancer related to prostate; slowing down or stopping the progression, aggravation, or deterioration of one or more symptoms of cancer; bringing about ameliorations of the symptoms of the cancer; reducing the severity or incidence of a cancer; or curing a cancer. A therapeutically effective amount may be administered prior to the onset of the cancer, for a prophylactic or preventive action. Alternatively, or additionally, the therapeutically effective amount may be administered after onset of the disease, for a therapeutic action. It is obvious that the quantity to be administered can be adapted by the man skilled in the art according to the subject to be treated, to the nature of the disease, etc. In particular, doses and regimen of administration may be function of the nature, of the stage and of the severity of the prostate cancer to be treated, as well as of the weight, the age and the global health of the subject to be treated, as well as of the judgment of the doctor.

The terms “RORy” and “ROR gamma” are used interchangeably and refer to either or both isoforms encoded by the RORC (RAR-related orphan receptor C) gene, namely RORγ (also referred to as RORyl or RORC1) and RORyt (also known as RORy2 or RORC2).

Throughout the disclosure, various aspects of the invention can be presented in a range format. It should be understood that the description in range format is for convenience and brevity and should not be constructed as an inflexible limitation on the scope of the invention. Accordingly, the description of a range should be considered to have specifically disclosed all the possible subranges as well as individual numerical values within that range, range values being included.

Compounds of Formula (I), (II) and (III)

The present invention relates to RORγ inhibitors, and more particularly to compounds of formula (I), (II) and (III) as disclosed in WO17044410, WO18160550 and WO18160547.

As used herein, the term “RORy inhibitors” designates compounds that inhibit retinoic acid receptor-related orphan receptor y (RORy) transcription, translation, stability, and/or activity. In certain embodiments, RORγ inhibitors bind to RORγ and inhibit the activity of the receptor. In some instances, inhibition of RORγ activity can include inhibition of recruitment of coactivators such as SRC-1 and/or SRC-3 to an androgen receptor (AR) ROR response element (RORE). In other instances, inhibition of RORy activity can include inhibition of transcription of the AR gene and/or a variant thereof such as AR-V7.

It is thus the purpose of the present invention to provide a new treatment for RORγ dependent cancer or metastasis, wherein an effective dose of a compound of formula (I), (II) and/or (III) is administered to a patient in need thereof. It is a particular purpose of the present invention to provide a new treatment for prostate cancer, which may be advantageously effective in the treatment of castration-resistant prostate cancer, wherein an effective dose of a compound of formula (I), (II) and/or (III) is administered to a patient in need thereof.

According to the invention, the treatment comprises or consists in the administration of a compound of formula (I), (II) and/or (III), wherein
n is 0, 1 or 2,
X is independently —N— or —CH—,
R1 is —C1-3 alkyl,
R2a and R2b are independently —H or —CH3,
R3a and R3b are independently —H, CH3 or —CH2CH3,
R4 is

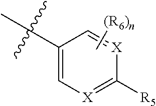

or thiazolyl, optionally substituted with —CF3,
R5 is halo, —CN, —CF3, oxadiazolyl, or oxadiazolyl, optionally substituted with —CH3,
R6 is —H, —OMe, halogen, —CH3 or —CF3.

The terms mentioned herein with prefixes such as for example C₁-C₃, can also be used with lower numbers of carbon atoms such as C₁-C₂. If, for example, the term C₁-C₃ is used, it means that the corresponding hydrocarbon chain may comprise from 1 to 3 carbon atoms, especially 1, 2 or 3 carbon atoms.

The term “alkyl” refers to a saturated, linear or branched aliphatic group. The term “(C₁-C₃)alkyl” more specifically means methyl, ethyl, propyl, or isopropyl.

The term “halogen”, or “halo”, corresponds to a fluorine, chlorine, bromine, or iodine atom, preferably a fluorine atom.

The expression “substituted by at least” means that the radical is substituted by one or several groups of the list.

In a preferred embodiment, the compound used in the treatment of prostate cancer is the compound of formula (III)

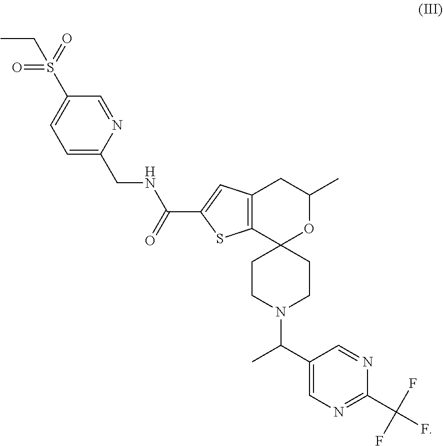 [SMILES: CCS(=O)(=O)c1ccc(CNC(=O)c2cc3c(s2)C2(CCN(C(C)c4cnc(C(F)(F)F)nc4)CC2)OC(C)C3)nc1]

A compound of the present invention may react to form pharmaceutically acceptable salts. Pharmaceutically acceptable salts and common methodology for preparing them are well known in the art. See, e.g., P. Stahl, et al. Handbook of Pharmaceutical Salts: Properties, Selection and Use, 2nd Revised Edition (Wiley-VCH, 2011); S. M. Berge, et al., “Pharmaceutical Salts,” Journal of Pharmaceutical Sciences, Vol. 66, No. 1, January 1977.

The compounds of the present invention may possess one or more asymmetric centers; such compounds can therefore be produced as individual (R)- or (S)-stereoisomers or as mixtures thereof. Unless indicated otherwise, the description or naming of a particular compound in the specification and claims is intended to include both individual enantiomers and mixtures, racemic or otherwise, thereof. The methods for the determination of stereochemistry and the separation of stereoisomers are well-known in the art (see discussion in Chapter 4 of “Advanced Organic Chemistry”, 4th edition J. March, John Wiley and Sons, New York, 2001), for example by synthesis from optically active starting materials or by resolution of a racemic form. Some of the compounds of the invention may have geometric isomeric centers (E- and Z-isomers). It is to be understood that the present invention encompasses all optical, diastereoisomers and geometric isomers and mixtures thereof that possess RORγ inhibitor activity.

Although the present invention contemplates all individual enantiomers, as well as mixtures of the enantiomers of said compounds including racemates, the preferred compounds of the invention are represented by (III) or pharmaceutically acceptable salts thereof.

The term “pharmaceutically acceptable salt,” as use herein, includes those salts which are, within the scope of sound medical judgment, suitable for use in contact with the tissues of humans and animals without undue toxicity, irritation, allergic response and the like and are commensurate with a reasonable benefit/risk ratio. Pharmaceutically acceptable salts are well known in the art. The salts can be prepared in situ during the final isolation and purification of the compounds of the invention or separately by reacting the free base group with a suitable organic or inorganic acid. Representative acid addition salts include acetate, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, lactobionate, lactate, laurate, lauryl sulfate, malate, maleate, malonate, methanesulfonate, 2-naphthalenesulfonate, nicotinate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, valerate salts, and the like. Representative alkali or alkaline earth metal salts include sodium, lithium, potassium, calcium, magnesium, and the like, as well as nontoxic ammonium, quaternary ammonium, and amine cations, including, but not limited to ammonium, tetramethylammonium, tetraethylammonium, methylamine, dimethylamine, trimethylamine, triethylamine, ethylamine, and the like.

The skilled artisan will also appreciate that the Cahn-Ingold-Prelog (R) or (S) designations for all chiral centers will vary depending upon the substitution patterns of the particular compound. The single enantiomers or diastereomers may be prepared beginning with chiral reagents or by stereoselective or stereospecific synthetic techniques. Alternatively, the single enantiomers or diastereomers may be isolated from mixtures by standard chiral chromatographic or crystallization techniques at any convenient point in the synthesis of compounds of the invention. Single enantiomers of compounds of the invention are a preferred embodiment of the invention.

The compounds of the present invention, or salts thereof, may be prepared by a variety of procedures known in the art, some of which are illustrated in WO17044410, WO18160550 and WO18160547.

Advantageously, the compound of the present invention has a half-maximal RORγ inhibitory concentration (IC50) value of from about 100 nM to about 100 μM, e.g., from about 100 nM to about 50 μM, from about 100 nM to about 25 μM, from about 100 nM to about 10 μM, from about 500 nM to about 100 μM, from about 500 nM to about 50 μM, from about 500 nM to about 25 μM, from about 500 nM to about 10 μM, from about 1 μM to about 100 μM, from about 1 μM to about 50 μM, from about 1 μM to about 25 μM, from about 1 μM to about 10 μM, or about 100 nM, 200 nM, 300 nM, 400 nM, 500 nM, 600 nM, 700 nM, 800 nM, 900 nM, 1 μM, 2 μM, 3 μM, 4 μM, 5 μM, 6 μM, 7 μM, 8 μM, 9 μM, or 10 μM. In some instances, the IC50 value for a specific compound is measured using an in vitro assay in cancer cells that have been incubated with said compound. The IC50 value can be determined based on the effect of the compound in inhibiting the survival of cancer cells such as cells from a cancer cell line or primary tumor cells. In other embodiments, the compound of the present invention has an inhibitor constant (Ki) that is essentially the same numerical value as the IC50 value, or is about one-half the value of the IC50 value.

Pharmaceutical Composition and Kit

The present invention further relates to a pharmaceutical composition for the treatment of a RORγ dependent cancer or metastasis, such as prostate cancer, pancreatic cancer, lung cancer, breast cancer, liver cancer, ovarian cancer, endometrial cancer, bladder cancer, colon cancer, lymphoma, and glioma comprising one or more compounds of the invention, or a pharmaceutically acceptable salt thereof, as an active ingredient, in combination with one or more pharmaceutically acceptable carriers, diluents, or excipients, and/or other drugs, in any dosage form, such as intravenous (IV), subcutaneous, intramuscular, intravesical, oral, or sustain release or local delivery. In a particular embodiment, the present invention relates to such pharmaceutical composition for the treatment of a prostate cancer, such as a castration-resistant prostate cancer.

“Pharmaceutically acceptable” or “therapeutically acceptable” includes a substance which does not interfere with the effectiveness or the biological activity of the active ingredients and which is not toxic to the hosts in the amounts used, and which hosts may be either humans or animals to which it is to be administered.

A “pharmaceutically acceptable, carrier, diluent or excipient,” as used herein, includes any ingredient other than the compounds described herein (for example, a vehicle capable of suspending or dissolving the active compound) and having the properties of being nontoxic and non-inflammatory in a patient. The excipient or carrier is selected on the basis of the mode and route of administration. Suitable pharmaceutical carriers, as well as pharmaceutical necessities for use in pharmaceutical formulations, are known. This may include, for example: antiadherents, antioxidants, binders, coatings, compression aids, disintegrants, dyes (colors), emollients, emulsifiers, fillers (diluents), film formers or coatings, flavors, fragrances, glidants (flow enhancers), lubricants, preservatives, printing inks, sorbents, suspensing or dispersing agents, sweeteners, or waters of hydration. Exemplary excipients include, but are not limited to: butylated hydroxytoluene (BHT), calcium carbonate, calcium phosphate (dibasic), calcium stearate, croscarmellose, cross-linked polyvinyl pyrrolidone, citric acid, crospovidone, cysteine, ethylcellulose, gelatin, hydroxypropyl cellulose, hydroxypropyl methylcellulose, lactose, magnesium stearate, maltitol, maltose, mannitol, methionine, methylcellulose, methyl paraben, microcrystalline cellulose, polyethylene glycol, polyvinyl pyrrolidone, povidone, pregelatinized starch, propyl paraben, retinyl palmitate, shellac, silicon dioxide, sodium carboxymethyl cellulose, sodium citrate, sodium starch glycolate, sorbitol, starch (corn), stearic acid, stearic acid, sucrose, talc, titanium dioxide, tween 80, vitamin A, vitamin E, vitamin C, and xylitol, micelles, liposomes; nanoparticles, PLA, PLGA, magnetic particles.

In a further embodiment, the composition further comprises one or more other therapeutic agents in combination with one or more compounds of the present invention.

According to an embodiment of the invention, the composition may further comprise an anticancer drug, e.g., an anti-androgen drug, a check-point inhibitor, a cytotoxic compound.

Non-limiting examples of anticancer drugs include anti-androgen drugs, chemotherapeutic agents, radiotherapeutic agents, antigen-specific or non-specific immunotherapeutic agents, adjuvants, endocrine therapies, tyrosine kinase inhibitors, and combinations thereof.

An “anti-androgen drug” includes anti-androgen compounds that alter the androgen pathway by blocking the androgen receptors, competing for binding sites on the cell's surface, or affecting or mediating androgen production. Anti-androgen drugs are useful for treating several diseases including, but not limited to, prostate cancer. Anti-androgen drugs include, but are not limited to, enzalutamide, abiraterone, bicalutamide, flutamide, nilutamide, apalutamide, finasteride, dutasteride, alfatradiol, and combinations thereof. A “chemotherapeutic agent” may be tamoxifen, a taxane (e.g., paclitaxel and/or docetaxel), or combinations thereof.

Chemotherapeutic agents are well known in the art and include, but are not limited to, anthracenediones (anthraquinones) such as anthracyclines (e.g., daunorubicin (daunomycin; rubidomycin), doxorubicin, epirubicin, idarubicin, and valrubicin), mitoxantrone, and pixantrone; platinum-based agents (e.g., cisplatin, carboplatin, oxaliplatin, satraplatin, picoplatin, nedaplatin, triplatin, and lipoplatin); tamoxifen and metabolites thereof such as 4-hydroxytamoxifen (afimoxifene) and N-desmethyl-4-hydroxytamoxifen (endoxifen); taxanes such as paclitaxel (taxol), docetaxel, cabazitaxel, hongdoushan A, hongdoushan B, hongdoushan C, baccatin I, baccatin II, and 10-deacetylbaccatin; alkylating agents (e.g., nitrogen mustards such as mechlorethamine (HN2), cyclophosphamide, ifosfamide, melphalan (L-sarcolysin), and chlorambucil); ethylenimines and methylmelamines (e.g., hexamethylmelamine, thiotepa, alkyl sulphonates such as busulfan, nitrosoureas such as carmustine (BCNU), lomustine (CCNLJ), semustine (methyl-CCN-U), and streptozoein (streptozotocin), and triazenes such as decarbazine (DTIC; dimethyltriazenoimidazolecarboxamide)); antimetabolites (e.g., folic acid analogues such as methotrexate (amethopterin), pyrimidine analogues such as fluorouracil (5-fluorouracil; 5-FU), floxuridine (fluorodeoxyuridine; FUdR), and cytarabine (cytosine arabinoside), and purine analogues and related inhibitors such as mercaptopurine (6-mercaptopurine; 6-MP), thioguanine (6-thioguanine; 6-TG), and pentostatin (2′-deoxycofonnycin)); natural products (e.g., vinca alkaloids such as vinblastine (VLB) and vincristine, epipodophyllotoxins such as etoposide and teniposide, and antibiotics such as dactinomycin (actinomycin D), bleomycin, plicamycin (mithramycin), and mitomycin (mitomycin Q); enzymes such as L-asparaginase; biological response modifiers such as interferon alpha); substituted ureas such as hydroxyurea; methyl hydrazine derivatives such as procarbazine (N-methylhydrazine; MIH); adrenocortical suppressants such as mitotane (o,ρ′-DDD) and aminoglutethimide; analogs thereof; derivatives thereof; and combinations thereof.

Radiotherapeutic agents are well known in the art and can comprise external-beam radiation therapy and/or internal radiation therapy. External beam radiation therapy delivers radioactive beams of high energy X-rays and/or gamma rays to a patient's tumor, whereas internal radiation therapy delivers radioactive atoms to a patient's tumor. Both external beam radiation therapy and internal radiation therapy are used to suppress tumor growth or kill cancer cells by delivering a sufficient quantity of radioactivity to the target site. In some embodiments, the radiotherapeutic agent comprises a radioactive atom and is complexed with a biologic or synthetic agent to increase delivery to the target site. Such biologic or synthetic agents are known in the art. Suitable radioactive atoms for use with the RORy inhibitors of the present invention include any of the radionuclides described herein, or any other isotope which emits enough energy to destroy a targeted tissue or cell. In some embodiments, radiotherapeutic agents may be coupled to targeting moieties, such as antibodies, to improve the localization of radiotherapeutic agents to cancerous cells.

In certain embodiments, the effective amount of a compound of the invention is an amount sufficient to sensitize an anti-androgen drug-resistant prostate cancer (e.g., castration-resistant prostate cancer) to anti-androgen drug treatment. The compound of the invention and anti-androgen drugs can be delivered to a subject via the same route of administration (e.g., orally or parenterally) or via different routes of administration (e.g., intravenously for compound of the invention and orally for anti-androgen drugs, or vice versa).

In some embodiments, the present invention provides a composition comprising a compound of the invention in combination with one or more endocrine therapies. Endocrine therapy is the manipulation of the endocrine system through the administration of specific hormones or drugs which inhibit or decrease the production or activity of targeted hormones or alter the gene expression pattern of targeted cells. Endocrine therapy is particularly useful in certain types of cancer, including prostate cancer. Any known hormone antagonist or modulator may be used in the present invention. Endocrine therapies useful in the present invention include, but are not limited to, aromatase inhibitors (e.g. letrozole), megestrol acetate, flutamide, tamoxifen, raloxifene, lasofoxifene, bazedoxifene, bazedoxifene/conjugated estrogens, and combinations thereof.

In another embodiment, the pharmaceutical compositions comprising one or more compounds of the present invention and the pharmaceutical compositions comprising one or more anticancer drugs are prepared as separate medicaments.

A compound of the present invention is preferably formulated as pharmaceutical compositions administered by a variety of routes, depending upon whether local or systemic treatment is desired and upon the area to be treated. Administration may be topical, parenteral, intravenous, intra-arterial, subcutaneous, intramuscular, intratumoral, intranasal, aerosol, by suppositories, oral administration, or delivered as sustained release injectable formulations or with catheters or particles with affinity for tumor cells or magnetic particles. Such pharmaceutical compositions and processes for preparing the same are well known in the art. See, e.g., Remington: The Science and Practice of Pharmacy (A. Gennaro, et al., eds., 22nd ed., Pharmaceutical Press, 2012).

The compositions can be formulated in a unit dosage form, each dosage containing, e.g., 0.1-500 mg of the active ingredients. For example, the dosages can contain from about 0.1 mg to about 50 mg, from about 0.1 mg to about 40 mg, from about 0.1 mg to about 20 mg, from about 0.1 mg to about 10 mg, from about 0.2 mg to about 20 mg, from about 0.3 mg to about 15 mg, from about 0.4 mg to about 10 mg, from about 0.5 mg to about 1 mg; from about 0.5 mg to about 100 mg, from about 0.5 mg to about 50 mg, from about 0.5 mg to about 30 mg, from about 0.5 mg to about 20 mg, from about 0.5 mg to about 10 mg, from about 0.5 mg to about 5 mg; from about 1 mg from to about 50 mg, from about 1 mg to about 30 mg, from about 1 mg to about 20 mg, from about 1 mg to about 10 mg, from about 1 mg to about 5 mg; from about 5 mg to about 50 mg, from about 5 mg to about 20 mg, from about 5 mg to about 10 mg; from about 10 mg to about 100 mg, from about 20 mg to about 200 mg, from about 30 mg to about 150 mg, from about 40 mg to about 100 mg, from about 50 mg to about 100 mg of the active ingredient, from about 50 mg to about 300 mg, from about 50 mg to about 250 mg, from about 100 mg to about 300 mg, or, from about 100 mg to about 250 mg of the active ingredient.

For preparing solid compositions such as tablets, the principal active ingredient is mixed with one or more pharmaceutical excipients to form a solid bulk formulation composition containing a homogeneous mixture of a compound of the present invention. When referring to these bulk formulation compositions as homogeneous, the active ingredient is typically dispersed evenly throughout the composition so that the composition can be readily subdivided into equally effective unit dosage forms such as tablets and capsules. This solid bulk formulation is then subdivided into unit dosage forms of the type described above containing from, for example, 0.1 to about 500 mg of the active ingredient of the present invention.

In a particular embodiment, the composition is formulated for oral administration in unit dosage form (e.g., a tablet, capsule, caplet, gelcap, or syrup). The term “unit dosage forms” includes physically discrete units suitable as unitary dosages for human subjects and other mammals, each unit containing a predetermined quantity of active material calculated to produce the desired therapeutic effect, in association with any suitable pharmaceutical excipient or excipients.

In another embodiment, the composition is formulated for topical administration (e.g., as a cream, gel, lotion, or ointment).

In another embodiment, the composition is formulated for intravenous administration or intratumoral administration (e.g., as a sterile solution free of particulate emboli and in a solvent system suitable for intravenous or intratumoral use).

It is another object of the present invention to provide a kit comprising a compound of formula (I), (II) and/or (III) and an anticancer drug, such as an anti-androgen drug (e.g., enzalutamide, abiraterone, and/or bicalutamide) and/or a chemotherapeutic agent (e.g., tamoxifen and/or a taxane such as docetaxel).

Kits with unit doses of the compounds described herein, e.g., in oral, rectal, transdermal, or injectable/parenteral doses (e.g., for intramuscular, intravenous, subcutaneous or intratumoral injection), are provided. In such kits, an informational package insert describing the use and attendant benefits of the composition for enhancing the therapeutic response in a subject with a RORγ dependent cancer or metastasis, particularly with a prostate cancer, may be included in addition to the containers containing the unit doses.

In some embodiments, the kit further comprises a label with instructions for administering the compound of formula (I), (II) or (III) and/or the anticancer drug to a subject.

Method of Treatment

It is an object of the present invention to provide a method of treatment of a RORγ dependent cancer or metastasis, wherein an effective amount of the compound according to the present invention, or any pharmaceutical salt thereof is administered to a patient in need thereof. It is a particular object of the present invention to provide a method of treatment of a prostate cancer, such as a castration-resistant cancer, wherein an effective amount of a compound according to the present invention, or any pharmaceutical salt thereof is administered to a patient in need thereof. The present invention also relates to the use of compound of formula (I), (II) or (III) in the manufacture of a medicament for treating a RORγ dependent cancer or metastasis, particularly a prostate cancer, such as a castration-resistant cancer.

RORγt signaling, often in response to IL-23/IL-23 receptor signaling, is required for the differentiation of naive CD4+ T-cells into a subset of T-cells designated Th17, which are distinct from the classical Th1 and Th2 cells, and supports their maintenance. Th17 cells produce interleukin-17A (IL-17) and IL-17F. In addition, Th17 cells produce a range of other factors known to drive inflammatory responses, including tumor necrosis factor-alpha (TNF-α), interleukin-6 (IL-6), GM-CSF, CXCL1 and CCL20. NK cells and innate lymphoid cells such as lymphoid tissue inducer (LTi)-like cells express IL-23 receptor and RORγt and produce IL-17 in response to stimulation and IL-23. There is substantial evidence that IL-23-responsive, RORγt, and IL-17-expressing cells are associated with cancer, particularly prostate cancer.

Androgen receptor (AR), a member of the nuclear receptor (R) superfamily, binds androgenic hormones testosterone or dihydrotestosterone in the cytoplasm and translocates to the nucleus. AR modulates, inter alia, transcription of target genes by binding to Androgen Response Elements (AREs) in the promoters of such target genes. AR is overexpressed and hyperactivated in castration-resistant prostate cancer (CRPC). RORy is a key driver of AR aberrant expression. RORy is overexpressed and amplified in metastatic tumors. Its overexpression confers CRPC cell growth. RORy recruits coactivators SRC-1 and -3 to an AR-RORE to stimulate AR gene transcription. Its specific antagonists strongly suppress the expression of AR and its variant AR-V7. RORy antagonists also markedly diminish genome-wide AR binding, H3K27ac marks and expression of the AR gene network. In vivo, the antagonists potently block tumor growth in multiple models and effectively sensitize CRPC tumors to enzalutamide, without overt toxicity.

RORy is a key player in CRPC by acting upstream of AR and a potential therapeutic target for advanced prostate cancers.

RORy is highly overexpressed in metastatic tumors of prostate cancer and functions as a key determinant of AR overexpression and aberrant signaling in CRPC cells and tumors. RORy-selective antagonists strongly inhibit AR gene expression, its genome-wide binding and growth of xenograft tumors.

Recently, it has been shown that RORy inhibitors may be of particular benefit in the treatment of prostate cancer.

By working on the development of new therapeutic treatments of prostate cancer, the applicant has discovered that compounds of formula (I), (II) and (III), which are valuable RORy inhibitors, are of particular interest. Indeed, the compounds of the present invention exhibit attractive drug candidate properties including affinity for the ROR gamma receptor, high specificity, potency and overall efficacy. Furthermore, the compounds of the present invention exhibit excellent drugability properties including adequate oral bio-availability, first-pass metabolism, half-lives, and pharmacokinetic properties including but not limited to good absorption, clearance, excretion and volume of distribution.

The present invention thus provides a method of treatment of prostate cancer, wherein a RORy inhibitor of formula (I), (II) or (III), or any pharmaceutical composition comprising such compound(s) is administered to a patient in need thereof.

In certain embodiments, the prostate cancer is an advanced stage prostate cancer selected from one or more of metastatic prostate cancer, drug-resistant prostate cancer (e.g., anti-androgen-resistant prostate cancer such as enzalutamide-resistant prostate cancer, abiraterone-resistant prostate cancer, bicalutamide-resistant prostate cancer, etc.; taxane-resistant prostate cancer; docetaxel-resistant prostate cancer; and the like), hormone refractory prostate cancer, castration-resistant prostate cancer (CRPC), metastatic castration-resistant prostate cancer, AR-V7-induced drug-resistant prostate cancer such as AR-V7-induced anti-androgen-resistant prostate cancer (e.g., AR-V7-induced enzalutamide-resistant prostate cancer), AKR1C3-induced drug-resistant prostate cancer such as AKR1C3-induced anti-androgen-resistant prostate cancer (e.g., AKR1C3-induced enzalutamide-resistant prostate cancer), and combinations thereof.

In some embodiments, the anticancer drug is selected from the group consisting of an anti-androgen drug, chemotherapeutic agent, radiotherapeutic agent, antigen-specific immunotherapeutic agent, endocrine therapy, tyrosine kinase inhibitor, and combinations thereof. In some instances, the anti-androgen drug is selected from the group consisting of enzalutamide, bicalutamide, arbiraterone, nilutamide, flutamide, apalutamide, finasteride, dutasteride, alfatradiol, and combinations thereof.

In other instances, the taxane is selected from the group consisting of paclitaxel, docetaxel, and combinations thereof.

In a non-limiting example, an effective amount of a compound according to formula (I), (II) or (III), or of a pharmaceutical composition containing said compound, includes an amount sufficient to alleviate the signs, symptoms, or causes of a prostate cancer, including castration-resistant prostate cancer. Thus, an effective amount can be an amount that slows or reverses tumor growth, increases mean time of survival, inhibits tumor progression or metastasis, or sensitizes a cancer cell to an anticancer drug to which it has become or is resistant.

Also, in another non-limiting example, an effective amount of a compound according to the invention or of a pharmaceutical composition according to the invention includes an amount sufficient to cause a substantial improvement in a subject having prostate cancer when administered to the subject. The amount will vary with the type of cancer being treated, the stage of advancement of the cancer, the type and concentration of composition applied, and if any, the amount of anticancer drug (e.g., anti-androgen drug) that is also administered to the subject.

In another non-limiting example, an effective amount of a compound according to the invention or of a pharmaceutical composition according to the invention can include an amount that is effective in enhancing the therapeutic activity of anticancer drugs such as anti-androgen drugs (e.g., bicalutamide, enzalutamide, arbiraterone, etc.) and/or chemotherapeutic agents (e.g., tamoxifen and/or taxanes such as docetaxel).

In another non-limiting example, an effective amount of a compound according to the invention or of a pharmaceutical composition according to the invention can be co-administered to a subject in combination with an effective amount of an anticancer drug at a therapeutically effective dose to treat the subject's cancer, as described herein. In another non-limiting example, an effective amount of a compound according to the invention or of a pharmaceutical composition according to the invention can be co-administered to a subject in combination with an effective amount of an anticancer drug to elicit an effective therapeutic response in the subject.

In a particular embodiment, the method for treating a prostate cancer in a subject comprises administering to the subject an effective amount of a compound according to the invention or of pharmaceutical composition according to the invention, in combination with an effective amount of an anticancer drug. In some embodiments, the prostate cancer is resistant to the anticancer drug.

In particular embodiments, the compound of the present invention enhances the therapeutic effect of the anticancer drug. For example, the compound of the present invention can reverse or reduce cancer cell resistance to the anticancer drug and/or sensitize cancer cells to the anticancer drug.

In some embodiments, the anticancer drug is selected from the group consisting of an anti-androgen drug, chemotherapeutic agent, radiotherapeutic agent, antigen-specific immunotherapeutic agent, endocrine therapy, tyrosine kinase inhibitor, and combinations thereof. In certain instances, the anti-androgen drug is selected from the group consisting of enzalutamide, bicalutamide, arbiraterone, nilutamide, flutamide, apalutamide, finasteride, dutasteride, alfatradiol, and combinations thereof. In other instances, the chemotherapeutic agent is tamoxifen, a taxane (e.g., paclitaxel and/or docetaxel), or combinations thereof.

As a non-limiting example, a subject with a prostate cancer that is resistant to treatment with an anti-androgen drug such as enzalutamide can be administered the anti-androgen drug with an amount of a compound according to the invention or of pharmaceutical composition according to the invention sufficient to reverse or reduce prostate cancer cell resistance to the anti-androgen drug and/or sensitize the prostate cancer cells to the anti-androgen drug.

As another non-limiting example, a subject with a prostate cancer that is resistant to treatment with a taxane such as docetaxel can be administered the taxane with an amount of a compound according to the invention or of pharmaceutical composition according to the invention sufficient to reverse or reduce prostate cancer cell resistance to the taxane and/or sensitize the prostate cancer cells to the taxane.

In a particular embodiment, the method of treatment includes co-administration of at least one compound or composition of the present invention and optionally additional compound or composition. As used herein, the term “co-administering” includes sequential or simultaneous administration of two or more structurally different compounds. For example, two or more structurally different pharmaceutically active compounds can be co-administered by administering a pharmaceutical composition adapted for oral administration that contains two or more structurally different active pharmaceutically active compounds.

As another example, two or more structurally different compounds can be co-administered by administering one compound and then administering the other compound. In some instances, the co-administered compounds are administered by the same route. In other instances, the co-administered compounds are administered via different routes. For example, one compound can be administered orally, and the other compound can be administered, e.g., sequentially or simultaneously, via intravenous, intrarectal, intraperitoneal, prostatic, paratumoral, intratumoral delivery or injection.

Regimen and Administration

The compounds according to the invention or the pharmaceutical composition according to the invention may be administered by any conventional route of administration. In particular, the compounds or the pharmaceutical composition of the invention can be administered by a topical, enteral, oral, parenteral, intranasal, intravenous, intra-arterial, intramuscular, subcutaneous, or intraperitoneal, or intratumoral administration and the like. In particular, the compound according to the invention or the pharmaceutical composition according to the invention can be formulated for a topical, enteral, oral, parenteral, intranasal, intravenous, intra-arterial, intramuscular, subcutaneous, intrarectal, intraperitoneal, prostatic or intratumoral administration and the like.

Particularly, in order to provide a localized therapeutic effect, specific prostatic administration routes are preferred. In particular, administration by injection directly within the prostate and more particularly within the tumor, is preferred.

In a particular embodiment, the compound according to the invention or the pharmaceutical composition according to the invention is administered by enteral or parenteral route of administration. When administered parenterally, the compound according to the invention or the pharmaceutical composition according to the invention is preferably administered by intravenous route of administration. When administered enterally, the compound according to the invention or the pharmaceutical composition according to the invention is preferably administered by oral route of administration.

For oral administration, the composition can be formulated into conventional oral dosage forms such as tablets, capsules, powders, granules and liquid preparations such as syrups, elixirs, and concentrated drops. Nontoxic solid carriers or diluents may be used which include, for example, pharmaceutical grades of mannitol, lactose, starch, magnesium stearate, sodium saccharine, talcum, cellulose, glucose, sucrose, magnesium, carbonate, and the like. For compressed tablets, binders, which are agents which impart cohesive qualities to powdered materials, are also necessary. For example, starch, gelatin, sugars such as lactose or dextrose, and natural or synthetic gums can be used as binders. Disintegrants are also necessary in the tablets to facilitate break-up of the tablet. Disintegrants include starches, clays, celluloses, algins, gums and crosslinked polymers. Moreover, lubricants and glidants are also included in the tablets to prevent adhesion to the tablet material to surfaces in the manufacturing process and to improve the flow characteristics of the powder material during manufacture. Colloidal silicon dioxide is most commonly used as a glidant and compounds such as talc or stearic acids are most commonly used as lubricants.

For transdermal administration, the composition can be formulated into ointment, cream or gel form and appropriate penetrants or detergents could be used to facilitate permeation, such as dimethyl sulfoxide, dimethyl acetamide and dimethylformamide.

For transmucosal administration, nasal sprays, rectal or vaginal suppositories can be used. The active compound can be incorporated into any of the known suppository bases by methods known in the art. Examples of such bases include cocoa butter, polyethylene glycols (carbowaxes), polyethylene sorbitan monostearate, and mixtures of these with other compatible materials to modify the melting point or dissolution rate.

Compound or pharmaceutical composition according to the invention may be formulated to release the active drug substantially immediately upon administration or at any predetermined time or time period after administration.

The present methods for treating RORγ dependent cancer or metastasis are carried out by administering a therapeutic for a time and in an amount sufficient to result in decreased tumor weight, decreased tumor growth, or decreased in other incidence of cancer progression or presence.

Particularly, the present methods for treating prostate cancer are carried out by administering a therapeutic for a time and in an amount sufficient to result in decreased tumor weight, decreased tumor growth, or decreased in other incidence of prostate cancer progression or presence.

The compound according to the invention or the pharmaceutical composition according to the invention may be administered as a single dose or in multiple doses.

Preferably, the treatment is administered regularly, preferably between every day and every month, more preferably between every day and every two weeks, more preferably between every day and every week, even more preferably the treatment is administered every day. In a particular embodiment, the treatment is administered several times a day, preferably 2 or 3 times a day, even more preferably 3 times a day.

The duration of treatment with the compound according to the invention or the pharmaceutical composition according to the invention is preferably comprised between 1 day and 20 weeks, more preferably between 5 days and 10 weeks, still more preferably between 5 days and 4 weeks, even more preferably between 5 days and 2 weeks. In a particular embodiment, the duration of the treatment is of at least 1 week. Alternatively, the treatment may last as long as tumoral cells persist.

The amount of compound according to the invention or of pharmaceutical composition according to the invention to be administered has to be determined by standard procedure well known by those of ordinary skills in the art. Physiological data of the patient (e.g. age, size, and weight) and the routes of administration have to be taken into account to determine the appropriate dosage, so as a therapeutically effective amount will be administered to the patient.

The amount and frequency of administration of the compositions can vary depending on, for example, what is being administered, the state of the patient, and the manner of administration.

In therapeutic applications of prostate cancer, compositions can be administered to a patient suffering from prostate cancer in an amount sufficient to relieve or least partially relieve the symptoms of the prostate cancer and its complications. The dosage is likely to depend on such variables as the type and extent of progression of the prostate cancer, the severity of the prostate cancer, the age, weight and general condition of the particular patient, the relative biological efficacy of the composition selected, formulation of the excipient, the route of administration, and the judgment of the attending clinician. Effective doses can be extrapolated from dose-response curves derived from in vitro or animal model test system. An effective dose is a dose that produces a desirable clinical outcome by, for example, improving a sign or symptom of the prostate cancer or slowing its progression.

The form of the pharmaceutical compositions, the route of administration and the dose of administration of the compound according to the invention, or the pharmaceutical composition according to the invention can be adjusted by the man skilled in the art according to the type and severity of the disease, and to the patient, in particular its age, weight, sex, and general physical condition.

The administration includes providing unit dosages of compounds or compositions set forth herein to a patient in need thereof. Administering includes providing effective amounts of compounds of formula (I), (II) or (III) for a specified period of time, e.g., for about 1, 2, 3, 4, 5, 6, 7, 8, 9, 10, 12, 13, 14, 15, 16, 17, 18, 19, 20, 21, 22, 23, 24, 25, 26, 27, 28, 29, 30, 31 or more days, or in a specified sequence, e.g., administration of one or more compounds of formula (I), (II) or (III) followed by the administration of one or more anticancer drugs, or vice versa.

The dosage of active agents administered is dependent on the subject's body weight, age, individual condition, surface area or volume of the area to be treated and on the form of administration. The size of the dose also will be determined by the existence, nature, and extent of any adverse effects that accompany the administration of a particular formulation in a particular subject. A unit dosage for oral administration to a mammal of about 50 to about 70 kg may contain between about 5 and about 500 mg, about 25-200 mg, about 100 and about 1000 mg, about 200 and about 2000 mg, about 500 and about 5000 mg, or between about 1000 and about 2000 mg of the active ingredient.

Typically, a dosage of the active compound(s) of the present invention is a dosage that is sufficient to achieve the desired effect. Optimal dosing schedules can be calculated from measurements of active agent accumulation in the body of a subject. In general, dosage may be given once or more of daily, weekly, or monthly. Persons of ordinary skill in the art can easily determine optimum dosages, dosing methodologies and repetition rates.

Optimum dosages, toxicity, and therapeutic efficacy of the compounds or compositions of the present invention may vary depending on the relative potency of the administered compounds or composition and can be determined by standard pharmaceutical procedures in cell cultures or experimental animals, for example, by determining the LD50 (the dose lethal to 50% of the population) and the ED50 (the dose therapeutically effective in 50% of the population). The dose ratio between toxic and therapeutic effects is the therapeutic index and can be expressed as the ratio, LDS o/ED50. Agents that exhibit large therapeutic indices are preferred. While agents that exhibit toxic side effects can be used, care should be taken to design a delivery system that targets such agents to the site of affected tissue to minimize potential damage to normal cells and, thereby, reduce side effects.

Optimal dosing schedules can be calculated from measurements of active ingredient accumulation in the body of a subject. In general, dosage is from about 1 ng to about 1,000 mg per kg of body weight and may be given once or more daily, weekly, monthly, or yearly. A typical composition of the present invention for oral or intravenous administration can be about 0.1 to about 10 mg of active ingredient per patient per day; about 1 to about 100 mg per patient per day; about 25 to about 200 mg per patient per day; about 50 to about 500 mg per patient per day; about 100 to about 1000 mg per patient per day; or about 1000 to about 2000 mg per patient per day. Exemplary dosages include, but are not limited to, about 10 mg, 20 mg, 25 mg, 50 mg, 75 mg, 100 mg, 200 mg, 300 mg, 400 mg, 500 mg, 600 mg, 700 mg, 800 mg, 900 mg, 1,000 mg, 1,250 mg, 1,500 mg, 2,000 mg, 2,500 mg, 3,000 mg, or more of the active ingredient per patient per day.

In some embodiments, a pharmaceutical composition of the present invention is administered, e.g., in a daily dose in the range from about 1 mg of compound per kg of subject weight (1 mg/kg) to about 1 g/kg. In another embodiment, the dose is a dose in the range of about 5 mg/kg to about 500 mg/kg. In yet another embodiment, the dose is about 10 mg/kg to about 250 mg/kg. In another embodiment, the dose is about 25 mg/kg to about 150 mg/kg. The daily dose can be administered once per day or divided into subdoses and administered in multiple doses, e.g., twice, three times, or four times per day. However, as will be appreciated by a skilled artisan, compositions described herein may be administered in different amounts and at different times. The skilled artisan will also appreciate that certain factors may influence the dosage and timing required to effectively treat a subject, including but not limited to the severity of the disease or malignant condition, previous treatments, the general health and/or age of the subject, and other diseases present. Moreover, treatment of a subject with a therapeutically effective amount of a composition can include a single treatment or, preferably, can include a series of treatments.

To achieve the desired therapeutic effect, compounds or agents described herein may be administered for multiple days at the therapeutically effective daily dose. Thus, therapeutically effective administration of compounds to treat prostate cancer in a subject may require periodic (e.g., daily) administration that continues for a period ranging from three days to two weeks or longer. Compositions set forth herein may be administered for at least three consecutive days, often for at least five consecutive days, more often for at least ten, and sometimes for 20, 30, 40 or more consecutive days. While consecutive daily doses are a preferred route to achieve a therapeutically effective dose, a therapeutically beneficial effect can be achieved even if the agents are not administered daily, so long as the administration is repeated frequently enough to maintain a therapeutically effective concentration of the agents in the subject. For example, one can administer the agents every other day, every third day, or, if higher dose ranges are employed and tolerated by the subject, once a week.

### Examples

The present invention will be described in greater detail by way of specific examples. The following examples are offered for illustrative purposes, and are not intended to limit the invention in any manner. Those of skill in the art will readily recognize a variety of noncritical parameters which can be changed or modified to yield essentially the same results.

General Method

Formulation:

Various formulations can be used for oral and parenteral administration

Dosing:

Doses are adapted for mice and human respectively.

Cell Line Selection Required for Both In Vitro and In Vivo Studies:

Cancer cell lines are associated with some advantages, including infinite proliferation ability and amenability to high-throughput drug screening. The three PCa cell lines, namely, DU145, PC-3, and LNCaP, are the most widely used cell lines in PCa research. At present, several PCa cell lines have been established from primary PCa tumors, PCa metastases, and PCa xenograft models. In addition, many sublines have been developed from these three classic PCa cell lines, especially the LNCaP cell line. Although the parental LNCaP cells are androgen-sensitive, sublines of these cells, such as LNCaP-abl and LNCaP-LTAD, established by depleting androgen from culture medium, are androgen-insensitive. Moreover, several drug-resistant PCa sublines have been developed by incubating the parental cell lines with antiandrogen or chemotherapeutic agents and could also be used in our models. Furthermore, as more cell lines are available, other experiments could be done with cell lines inducing metastatic castration-resistant prostate cancer, AR-V7-induced drug-resistant prostate cancer such as AR-V7-induced anti-androgen-resistant prostate cancer (e.g., AR-V7-induced enzalutamide-resistant prostate cancer), AKR1C3-induced drug-resistant prostate cancer, such as AKR1C3-induced anti-androgen-resistant prostate cancer (e.g., AKR1C3-induced enzalutamide-resistant prostate cancer) See references 1-6.

| Name | Pathology | Origin | Race | Pretreatment | AR | PSA | First Report Year |
|---|---|---|---|---|---|---|---|
| DU-145 | Adeno | metastasis | Caucasian | none | − | − | 1975 |
| PC-3 | Adeno | metastasis | Caucasian | none | − | − | 1979 |
| LNCaP | Adeno | metastasis | Caucasian | none | + | + | 1980 |
| 22Rv1 | Adeno | xenograft | Caucasian | none | + | + | 1999 |
| | | tumor from | | | | | |
| | | primary | | | | | |
| | | tumor | | | | | |

In Vitro Studies:

Cell viability, apoptosis and growth assays, and colony formation:

For instance, for cell viability, prostate cancer cells are seeded in 96-well plates at 1500-2500 cells per well 20 (optimum density for growth) in a total volume of 100 pl media. Serially diluted compounds in 100 pl of media are added to the cells 12 hours later. After 4 days of incubation, CellTiter GLO reagents (Promega) are added and luminescence is measured on GLOMAX microplate luminometer (Promega), according to the manufacturer's instructions. All experimental points are set up as sextuplicate as biological replication and the entire 25 experiments are repeated three times. The data can be presented as percentage of viable cells with vehicle treated cells set as 100. The estimated in vitro ICso values are calculated using graphic software.

For apoptosis, Terminal deoxynucleotidyl transferase-mediated dUTP nick end labeling (TUNEL) can be performed by using in situ cell death detection kit (Roche). The results are expressed as a percentage of apoptotic cell number/total cell number. Caspase-3/7 activity can be measured using a luminescent caspase Glo 3/7 assay kit (Promega Corporation, Madison, USA) following the manufacturer's instructions. For cell growth, cells are seeded in 6-well plates at 2 & 10′ per well and treated as indicated. Total viable cell numbers can be counted using a Coulter cell counter. For colony formation, 800 cells are seeded in a well of 6-well plates and cultured for 14 days with the medium changed every 3 days. When the cell clone grows visible, the medium is removed and the cells are fixed with 10% formalin for 10 minutes. Then the plates are washed with PBS for two times and the cell colonies are stained with 0.2% crystal violet (in 10% formalin) for 15 minutes. The numbers of cell colonies are counted after washed 5 times by PBS. The above assays are performed in triplicates and the entire experiments are repeated three times.

In Vivo Models:

Several types of murine prostate cancer models are available: cell line or human derived xenograft or orthotopic models (PDX) and human derived 3D models (PDC and PDO). Each model has its own advantages and limitations in terms of study design and expected outcome. Traditional cell lines are usually established from metastatic lesions and basically adapted to 2-dimensional monolayer culture. In contrast to cell line platform that have been traditionally used with historical cell lines comprising DU-145, LNCaP and the likes, recently developed platforms of patient-derived xenograft (PDX) and patient-derived cancer cells (PDCs) in 3-dimensional organoids/spheroids have advantages, as they often retain the characteristics of the original tumor including tumor heterogeneity and complexity. PDX models have advantages, including microenvironment, but limitations due to immunodeficient host background. PDCs can be also applied to PDX models with improved success rates for tumor formation or secondary PDC organoids/spheroids can be regenerated from PDX models vice versa. Organoid/spheroid culture and xenograft models derived from PCa cell lines can also be generated, although these platforms have limitations as they are apart from the actual behavior of clinical prostate cancer without original clinical data.

As none of the model cited above is fully representative of mCRPC, we will be therefore measuring the anti-tumor activity of our compounds in 3 types of models: traditional xenograft cell lines, (subcutaneous and orthotopic), patient-derived cell lines (subcutaneous and orthotopic), and 3-D organoid models. For most models, when possible, compounds are administrated by oral or parenteral routes.

1) Xenograft Cell Lines Model (Subcutaneous or Orthotopic)

Method:

This example describes two studies using in BALB/c nude mice to test the effect of compound III to be administered systemically (oral or parenteral administration).

Male mice of 6-8 weeks of age with body weights in the range of 18-22 grams are selected at the onset of the study. Several phenotypically distinct human prostate carcinoma cell lines are selected for xenograft formation. PC-3, 22Rv1, DU-145, LNCaP are obtainable from commercial sources.

Study Design:

PC-3, 22Rv1, DU-145, LNCaP or any other pertinent tumor cells are maintained in a monolayer culture under standard cell culture conditions. Cells growing in an exponential phase are harvested for tumor inoculation. Each mouse is inoculated at both the left and right flank regions with tumor cells (1×107) in 0.1 ml of PBS mixed with matrigel (1:1) for tumor development. The treatments are started when the mean tumor size of left or right flank reached about 100-150 mm3.

Before grouping and treatment, all animals are weighed, and the tumor volumes are measured every other day using a caliper. Since the tumor volume can impact the effectiveness of any given treatment, tumor volume is used as numeric parameter to randomize selected animals into specified groups.

Mouse Assay:

Each test group contains five mice as follows:

1) Vehicle-5 μl/tumor administered by parenteral injection on day 0 and 5 μl/tumor QW thereafter;

2) Compound III: 30 μl/tumor administered orally on day 0 (loading dose) and 10 μl/mouse QD thereafter (daily or testing dose);

3) SRL weekly-5 μl/tumor administered by parenteral injection on day 0 and 5 μl/tumor QW thereafter; and

4) SRL biweekly-5 μl/tumor administered by parenteral injection on day 0 and 5 μl/tumor BIW thereafter.

After tumor cells are inoculated, the mice are checked daily for morbidity and mortality. Body weights and tumor volumes are measured twice weekly. Blood PSA levels are measured on days 0, 12 and 22. Tumor volumes are measured in two dimensions using a caliper, and the volume is expressed in cubic millimeters (mm3) using the formula:

V=0.5a×b2

where a and b are the long and short diameters of the tumor, respectively. Tumor weight as measured at study termination. The study is terminated when the mean tumor burden (left+right) in each group reaches a value of 2000 mm3 or one week after the final dose (day 27).

Tumor growth inhibition (TGI), which is an indication of the effectiveness of a treatment regimen, is determined using the following formula:

TGI (%)=100×(1−TRTV/CRTV)

where TRTV and CRTV are the mean relative tumor volume of the treated and control groups, respectively, on a given day.

RTV (relative tumor volume) was calculated using the following formula:

RTV=Vt/V0

where Vt=tumor volume of the drug-treated group on a given day of the study, and V0=tumor volume of the drug-treated group on the initial day of dosing (day 0).

2) Patient Derived Xenograft (PDX) Oral and Parenteral Administrations (Subcutaneous and Orthotopic Model)

Patient-derived xenografts (PDXs) are an important preclinical cancer model for overcoming limitations associated with the use of cancer cell lines and allow investigators to obtain preclinical results that more accurately reflect clinical responses in patients. This is because PDXs grown in immunocompromised mice retain the key molecular aberrations present in patient tumors, including mutations, structural genomic events, epigenetic features, and gene expression programs, which drive their three-dimensional growth. The transplantation of human PCa tissues in athymic mice was initially associated with a very poor success rate. Meanwhile, recent technical innovations, including the use of novel highly immunodeficient mice, co-injection of PCa tissues with extracellular matrix (ECM), and transplantation into renal capsules, have improved the success rate of PDX transplantation. At present, several PCa PDX models have been developed by multiple research groups worldwide.

Study Design

Similar to above described in the section cancer cell derived xenograft but special attention has to be given to both the quality of tumor tissue and its site of implantation.

Traditional xenograft sites for PDX models include subcutaneous, renal capsule, and orthotopic transplantation. The ideal transplant site is believed to be the orthotopic site (includes the primary site of the tumor and the metastatic site of the tumor), which provides the tumor the same anatomical microenvironment. However, for Pca, the orthotopic transplantation operation is challenging because of the limited capacity of the mouse prostate and considerable damage to host mice. Therefore, the subrenal capsule has been suggested as a suitable site for Pca xenograft. It can significantly improve the success rate of a PDX model, which may be due to the rich vascular structure of the subrenal capsule itself, providing sufficient nutrients, hormones, and oxygen for early tumor tissue growth. Compared with the other two sites, the take rate of tumor tissue by the subrenal capsule can exceed 90%, and the histopathological structure, marker expression, and androgen sensitivity of the patient's tumor are faithfully reproduced. Moreover, subrenal capsule xenograft has facilitated the dissection of cancer cell-microenvironment interactions in some cells with metastatic potential in primary tumors of patients, providing a valuable tool for the study of Pca metastasis mechanisms. In particular, combined with SVM technology, subrenal capsule xenograft can partially simulate the tumor microenvironment in orthotopic grafts and maintain the original tumor integrity.

Tumor Tissue

High-quality Pca tissue is equally vital for establishing a PDX model. The following features of original cancer tissue will maximize the take rate of the xenograft: (I) a proportion of at least 50% of cancer; (II) high-quality undamaged tissue; and (III) the presence of proliferating cancer cells, such as those containing Ki67+ cells. Laboratory tumor samples are usually obtained from Pca biopsy, radical prostatectomy, or transurethral resection of the prostate. Samples obtained by different treatments might be in different stages of the disease, and the state of the tissue varies greatly. Pathologists need to assess the Pca tissue; curtail it precisely to reduce fat, necrosis, and other tissues; and minimize the time of ischemia after the sample is removed. Then, it should be transplanted into the mice as quickly as possible to ensure tissue vitality. Simultaneously, both researchers and surgeons should be in close communication with each other to obtain continuous, clinically relevant information, including patient tumor grade, androgen dependence, PSA and AR expression, metastasis, treatment, and recurrence.

3) Patient Derived Organoid Models

PDOs, grown in 3D culture with dissociated cells, is a novel pre-clinical model system in oncology that allows ex vivo propagation of tumors from individual patients. This 3D culture system for prostate tissue commonly use Matrigel for the extracellular matrix (ECM) component with a liquid medium overlay to embed prostate epithelial cells. It would more closely reflect the natural behave of PCa tissue in vivo, rendering its in vitro propagation easier. When prostatic tissues are maintained in a complex matrix environment, and its utilization for prediction of therapeutic response is much more reliable.

PDO experiments can also be performed using PDX-derived tissues, allowing long-term propagated tumors to be studied in a high-throughput manner. Furthermore, PDOs can be transplanted back into mice to be grown as PDXs, allowing long-term growth in vivo. Because of their high culture take rate, it allows side-by-side comparison to evaluate the translational potential of this model system to the patient.

4) Immunochemistry:

Immunochemistry should be performed at the conclusion of each model described above. Specifically, tumors from mice of all groups are harvested upon sacrifice at the end of the study and frozen in liquid nitrogen. The frozen tumors are sectioned and stained with antibodies attached to chromophores or enzymes to permit visualization of antibody binding by standard staining protocols. Staining (brown) is assessed in cells containing nuclei (blue) to rule out non-specific background noise. Statistical analysis of differences among various groups is performed using unpaired t test. P values for all groups were calculated versus the vehicle group, and the differences with a p value <0.05 should be considered statistically significant: *p<0.05, **p<0.01, ***p<0.001.

In addition, the impact of test compounds on molecular markers of tumor cell function should be assessed. Levels of Ki-67 are measured to assess tumor cell proliferation. Ki-67 is a nuclear protein that is universally expressed among proliferating cells (G1, S, G2, and mitosis) but is absent in quiescent cells (G0).

Levels of cleaved caspase-3 (CASP3) should be measured to assess tumor cell apoptosis. CASP3 is a member of the cysteine-aspartic acid protease family. CASP3 is a zymogen that is activated in apoptotic cells upon cleavage by an initiator caspases.

Levels of epithelial-cadherin (E-cadherin) should be measured to assess tumor cell metastasis. E-cadherin is a calcium-dependent cell-cell adhesion glycoprotein. Downregulation of E-cadherin decreases cellular adhesion in tissues, which is associated with an increase in cell motility and metastasis.

### REFERENCES

1) Current mouse and cell models in prostate cancer research; Xinyu Wu, Shiaoching Gong, Pradip Roy-Burman, Peng Lee and Zoran Culig Endocr Relat Cancer. 2013 August; 20(4): 10.
2) Development of patient-derived xenograft models of prostate cancer for maintaining tumor heterogeneity Changhong Shi, Xue Chen, and Dengxu Tan Transl Androl Urol. 2019 October; 8(5): 519-528.
3) Development and Characterization of a Spontaneously Metastatic Patient-Derived Xenograft Model of Human Prostate Cancer Tobias Lange et al; Sci Rep. 2018 Dec. 3; 8(1):17535.
4) Targeting castration-resistant prostate cancer with a novel RORγ antagonist elaiophylin, Jianwei Zheng et al; Acta Pharmaceutica Sinica B, July 2020,
5) Mouse models of prostate cancer: picking the best model for the question, Magdalena M Grabowska, David J DeGraff, Xiuping Yu, Ren Jie Jin, Zhenbang Chen, Alexander D Borowsky, Robert J Matusik; Cancer Metastasis Rev 2014 September; 33(2-3):377-97
6) Application of Prostate Cancer Models for Preclinical Study: Advantages and Limitations of Cell Lines, Patient-Derived Xenografts, and Three-Dimensional Culture of Patient-Derived Cells, Takeshi Namekawa, Kazuhiro Ikeda, Kuniko Horie-Inoue, Satoshi Inoue Cells 2019 Jan. 20; 8(1):74.

1-16. (canceled)

17. A method of treating of a RORγ dependent cancer or metastasis, wherein an effective amount of the compound according to Formula (I), a pharmaceutical salt thereof, or a composition comprising said compound or pharmaceutical salt is administered to a subject in need of treatment, 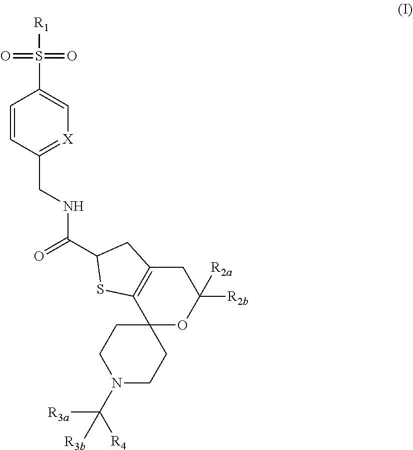
wherein
n is 0, 1 or 2,
X is independently —N— or —CH—,
R1 is —C1-3 alkyl,
R2a and R2b are independently —H or —CH3,
R3a and R3b are independently —H, CH3 or —CH2CH3,
R4 is 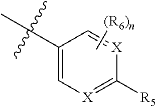
or thiazolyl optionally substituted with —CF3,
R5 is halo, —CN, —CF3, oxadiazolyl, or oxadiazolyl optionally substituted with CH3,
R6 is —H, —OMe, halo, —CH3 or —CF3.

18. The method of claim 17, wherein the compound is of formula (II), 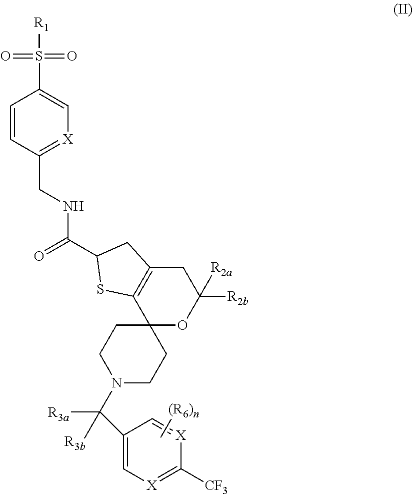
wherein
n is 0 or 1,
X is independently —N— or —CH—,
R1 is —C1-3 alkyl,
R2a and R2b are independently —H or —CH3,
R3a and R3b are independently —H, CH3 or —CH2CH3,
R6 is —H, —OMe, halo, —CH3 or —CF3.

19. The method of claim 17, wherein the compound is of formula (III),

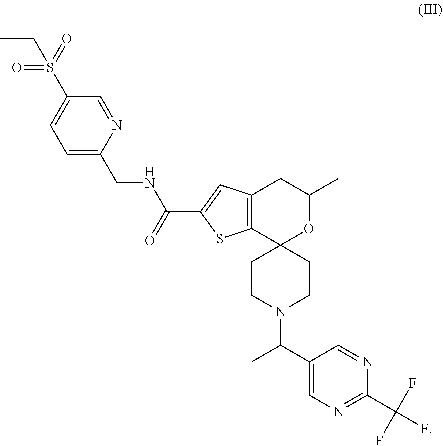 [SMILES: CCS(=O)(=O)c1ccc(CNC(=O)c2cc3c(s2)C2(CCN(C(C)c4cnc(C(F)(F)F)nc4)CC2)OC(C)C3)nc1]

20. The method of claim 17, said method further comprising the administration of an effective amount of an anticancer drug.

21. The method of claim 20, wherein the anticancer drug is selected from the group consisting of an anti-androgen drug, a chemotherapeutic agent, a radiotherapeutic agent, an antigen-specific immunotherapeutic agent, an endocrine therapy, a tyrosine kinase inhibitor, and combinations thereof.

22. The method of claim 17, wherein the RORγ dependent cancer is selected from the group consisting of prostate cancer, breast cancer, pancreatic cancer, gastric cancer, hepatocellular carcinoma (HCC), lung cancer, liver cancer, ovarian cancer, endometrial cancer, bladder cancer, colon cancer, lymphoma, and glioma.

23. The method of claim 22, wherein the RORγ dependent cancer is a prostate cancer.

24. The method of claim 23, wherein the prostate cancer is a castration-resistant prostate cancer.

25. The method of claim 22, wherein the breast cancer is Triple-Negative Breast Cancer (TNBC).

26. The method of claim 21, wherein the anti-androgen drug is selected from the group consisting of enzalutamide, bicalutamide, arbiraterone, nilutamide, flutamide, apalutamide, finasteride, dutasteride, alfatradiol, and combinations thereof and the chemotherapeutic agent is selected from the group consisting of tamoxifen, a taxane, or combinations thereof.

27. A kit for use in the treatment of a RORγ dependent cancer or metastasis comprising a compound of formula (I), (II) and/or (III) or a pharmaceutically acceptable salt thereof, and an anticancer drug.

28. The kit for use of claim 27, wherein the anticancer drug is selected from the group consisting of an anti-androgen drug, a chemotherapeutic agent, a radiotherapeutic agent, an antigen-specific immunotherapeutic agent, an endocrine therapy, a tyrosine kinase inhibitor, and combinations thereof.

29. The kit for use of claim 27, further comprising a label with instructions for administering the compound of formula (I), (II) or (III) or a pharmaceutically acceptable salt thereof, and/or the anticancer drug to a subject in need of RORγ dependent cancer treatment.

30. The kit for use of claim 28, wherein the anti-androgen drug is selected from the group consisting of enzalutamide, bicalutamide, arbiraterone, nilutamide, flutamide, apalutamide, finasteride, dutasteride, alfatradiol, and combinations thereof and the chemotherapeutic agent is selected from the group consisting of tamoxifen, a taxane, or combinations thereof.
